(19) United States
(12) Patent Application Publication
(10) Pub. No.: US 2023/0215528 A1
(43) Pub. Date: Jul. 6, 2023

(54) CONDITION INFORMATION GENERATION DEVICE

(71) Applicant: NIHON KOHDEN CORPORATION, Tokyo (JP)

(72) Inventors: Takayuki SUGIYAMA, Tokorozawa-shi (JP); Akihiko Yoshimura, Tokorozawa-shi (JP)

(73) Assignee: NIHON KOHDEN CORPORATION, Tokyo (JP)

(21) Appl. No.: 18/086,900

(22) Filed: Dec. 22, 2022

(30) Foreign Application Priority Data
Jan. 5, 2022 (JP) 2022-000424

(51) Int. Cl. G16H 10/60; G16H 50/30

(52) U.S. Cl. CPC G16H 10/60; G16H 50/30

(57) ABSTRACT

A condition information generation device includes an acquisition unit configured to acquire physiological information of a patient, a storage unit in which a determination method for determining a condition of the patient is stored, and a generation unit configured to generate condition information on the condition of the patient based on the acquired physiological information and the determination method. The generation unit determines whether there is missing physiological information in the acquired physiological information with respect to physiological information normally used when the determination method is executed, and generates missing information indicating that there is missing physiological information in a case where there is missing.

### CROSS-REFERENCE TO RELATED APPLICATIONS

The present application claims priority from Japanese Patent Application No. 2022-000424, filed on Jan. 5, 2022, the entire content of which is incorporated herein by reference.

### TECHNICAL FIELD

The presently disclosed subject matter relates to a condition information generation device.

### BACKGROUND ART

As a method of determining a change in a condition of a patient, for example, a method such as national early warning score (NEWS) or quick SOFA (qSOFA) is known. JP2020-089595A discloses a condition change determination device capable of displaying information based on a score calculated by a condition determination method such as NEWS or qSOFA.

When the condition of a patient is determined by a condition determination method such as NEWS or qSOFA, even if a part of necessary information is insufficient, for example, a score may be calculated based on acquired physiological information, and the condition of the patient may be determined based on the calculated score. However, the score calculated when all the necessary information has not been acquired is lower in accuracy than a score calculated when all the necessary information has been acquired. In the device disclosed in JP2020-089595A, it is not possible to easily know whether the calculated score is a score calculated in a state in which all necessary information is acquired.

An object of the presently disclosed subject matter is to provide a condition information generation device capable of easily grasping condition information on a condition of a patient and accuracy of the condition information.

### SUMMARY

According to a first aspect of the presently disclosed subject matter, there is provided a condition information generation device including an acquisition unit configured to acquire physiological information of a patient, a storage unit in which a determination method for determining a condition of the patient is stored, and a generation unit configured to generate condition information on the condition of the patient based on the acquired. physiological information and the determination method. The generation unit determines whether there is missing physiological information in the acquired physiological information with respect to physiological information normally used when the determination method is executed, and generates missing information indicating that there is missing physiological information in a case where there is missing.

According to the condition information generation device configured as described above, the generation unit generates the condition information based on the acquired physiological information and the determination method. In addition, the generation unit determines whether there is missing physiological information in the acquired physiological information with respect to the physiological information normally used when the determination method is executed. Then, when there is missing physiological information, the generation unit generates the missing information indicating that there is missing. That is, the generation unit generates the condition information and the missing information when there is missing physiological information in the acquired physiological information with respect to the physiological information normally used when the determination method is executed.

### BRIEF DESCRIPTION OF DRAWINGS

FIG. 1 is a block diagram of a condition information generation device according to an aspect of the presently disclosed subject matter.

FIG. 2 is a diagram illustrating a reference value of NEWS, which is one of condition determination methods.

FIG. 3 is a diagram illustrating a normalization table in NEWS.

FIG. 4 is a diagram illustrating a normalization table for normalizing each piece of physiological information.

FIG. 5 is a flowchart of a process performed by the condition information generation device.

FIG. 6 is a diagram illustrating an example of a display screen.

FIG. 7 is a diagram illustrating an example of the display screen.

FIG. 8 is a diagram illustrating an example of the display screen.

FIG. 9 is a diagram illustrating an example of the display screen.

FIG. 10 is a diagram illustrating an example of the display screen.

FIG. 11 is a diagram illustrating an example of the display screen.

FIG. 12 is a diagram illustrating an example of the display screen.

FIG. 13 is a diagram illustrating an example of the display screen.

FIG. 14 is a diagram illustrating an example of the display screen.

### DESCRIPTION OF EMBODIMENTS

Hereinafter, an example of the presently disclosed subject matter will be described with reference to the drawings.

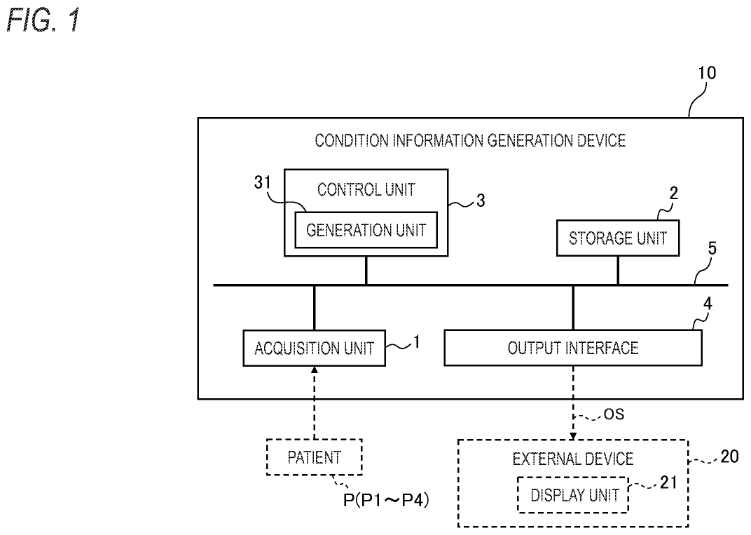

First, a condition information generation device 10 according to the presently disclosed subject matter will be described with reference to FIG. 1. As illustrated in FIG. 1, the condition information generation device 10 includes an acquisition unit 1, a storage unit 2, a control unit 3, and an output interface 4. These constituent elements are connected to each other via a bus 5 so as to be able to communicate with each other.

The acquisition unit 1 is configured to acquire physiological information of a patient P. The acquisition unit 1 may include acquisition circuitry configured to acquire physiological information of the patient P. The physiological information acquired by the acquisition unit 1 includes a continuous measurement parameter that is measured temporally continuously in a predetermined time range and a discontinuous measurement parameter that is measured at any timing. The continuous measurement parameter is a parameter for which data is continuously measured from a sensor attached to the patient P. The continuous measurement parameters are, for example, percutaneous arterial oxygen saturation (SpO₂), a heart rate, a respiration rate, and the like. The predetermined time range can be appropriately set by a medical worker. Further, the medical worker is, for example, a ward nurse, a certified nurse, a doctor, and the like. The discontinuous measurement parameter is a parameter that is measured in a spot manner from the sensor attached to the patient P, such as a non-invasive blood pressure measurement or a body temperature measurement by a round diagnosis. The discontinuous measurement parameters are, for example, oxygen assistance, a body temperature, a systolic blood pressure, a consciousness level, and the like.

The storage unit 2 stores a condition determination method for determining the condition of the patient P, a reference value and an allowable range defined by a medical guideline or the like related to the condition determination method, a normalization table for normalizing each determination result, the physiological information of the patient P, and the like. The storage unit 2 may include at least one of a magnetic recording medium (for example, a flexible disk, a magnetic tape, or a hard disk drive), a magneto-optical recording medium (for example, a magneto-optical disk), and a semiconductor memory (for example, a mask ROM, a programmable ROM (PROM), an erasable PROM (EPROM), a flash ROM). Examples of the condition determination method include qSOFA, NEWS, Sequential (Sepsis-related) Organ Failure Assessment (SOFA), acute physiology and chronic health evaluation 2 (APACHE 2), bedside shivering assessment scale (BSAS), and national institutes of health stroke scale (NIIISS). In addition, the condition determination method stored in the storage unit 2 may include, for example, a unique condition determination method set based on NEWS or the like, in addition to these above condition determination methods.

The control unit 3 includes a generation unit 31. In addition, the control unit 3 includes a memory and a processor as a hardware configuration. The memory includes, for example, a read only memory (ROM) in which various computer programs and the like are stored, a random access memory (RAM) having a plurality of work areas in which various computer programs and the like executed by the processor are stored, and the like. The processor is, for example, a central processing unit (CPU), and is configured to load a computer program designated from various computer programs incorporated in the ROM on the RAM and execute various processes in cooperation with the RAM. For example, the control unit 3 controls a processor of the control unit 3 to execute a computer program in cooperation with the RAM, thereby realizing the process of the generation unit 31.

The computer program may be stored in, for example, various types of non-transitory computer readable medium and supplied to the computer. The non-transitory computer readable medium includes any type of tangible storage medium. Examples of the non-transitory computer readable medium include a magnetic recording medium (for example, a flexible disk, a magnetic tape, or a hard disk drive), a magneto-optical recording medium (for example, a magneto-optical disk), a CD-read only memory (CD-ROM), a CD-R, a CD-R/W, and a semiconductor memory (for example, a mask ROM, a programmable ROM (PROM), an erasable PROM (EPROM), a flash ROM, or the like).

The generation unit 31 is configured to generate the condition information based on the physiological information of the patient P acquired by the acquisition unit 1 and the condition determination method, the normalization table, and the like stored in the storage unit 2. The condition information is information on the condition of the patient P. The information on the condition of the patient P is, for example, numerical information such as a score calculated based on the physiological information and the condition determination method, condition level information of the patient P generated based on the numerical information or the physiological information and the normalization table, and the like. The condition level information is, for example, a grade or the like indicating a degree of the condition of the patient P. The generation unit 31 transmits the generated condition information to the storage unit 2.

The generation unit 31 can determine whether there is missing physiological information in the physiological information normally used when the condition determination method is executed. The term “missing” means that all or a part of physiological information normally used when the condition determination method is executed is missing. Specifically, in a case where the acquisition portion 1 cannot acquire all or a part of the physiological information normally used when the condition determination method is executed, a case where all or a part of the physiological information acquired by the acquisition portion 1 among the physiological information normally used when the condition determination method is executed is not suitable for executing the condition determination method, or the like, the generation unit 31 determines that there is missing physiological information in the physiological information normally used when the condition determination method is executed. The case where the acquisition unit 1 cannot acquire all or a part of the physiological information normally used when the condition determination method is executed includes a case where the acquisition of the physiological information is not executed, a case where the acquisition unit 1 cannot acquire the physiological information due to a communication failure or the like although the measurement of the physiological information is performed, and the like, The case where all or a part of the physiological information acquired by the acquisition unit 1 among the physiological information normally used when the condition determination method is executed is not suitable for executing the condition determination method is, for example, a case where noise is included in the acquired physiological information.

When the generation unit 31 determines that there is missing physiological information in physiological information normally used when the condition determination method is executed, the generation unit 31 generates missing information indicating that there is missing. For example, the physiological information normally used when executing NEWS is SpO₂, oxygen assistance, a body temperature, a systolic blood pressure, a heart rate, a consciousness level, and a respiration rate, and when the body temperature is missing among them, the generation unit 31 determines that the body temperature is missing. Then, based on the determination, the generation unit 31 generates the missing information indicating that the body temperature is missing in the physiological information normally used when executing the NEWS.

When generating the missing information, the generation unit 31 can also generate specific information for specifying physiological information corresponding to the generated missing information. The specific information is information for specifying at least one of the number and the type of physiological information corresponding to the generated missing information. For example, when the body temperature is missing in the physiological information normally used when executing NEWS, the generation unit 31 generates specific information for specifying that the number of pieces of missing physiological information is one and the type of missing physiological information is the body temperature.

When it is determined that there is missing physiological information in the physiological information normally used when the condition determination method is executed, the generation unit 31 generates the condition information based on the physiological information acquired by the acquisition unit 1, complementary physiological information for complementing the missing physiological information, and the condition determination method. The complementary physiological information is, for example, a zero value for making the value of the score calculated based on the condition determination method zero, an average value of the physiological information acquired so far, the latest physiological information among the physiological information acquired so far, a maximum value of the physiological information acquired so far, a minimum value of the physiological information acquired so far, and the like. When the generation unit 31 generates the condition information based on the acquired physiological information, the complementary physiological information, and the condition determination method, the missing information generated by the generation unit 31 includes complementary type information on a type of complement. The complementary type information is information on the type of complement of the missing physiological information. For example, when the generation unit 31 generates the condition information on an assumption that the missing physiological information has a score of a zero value, the complementary type information is information indicating that the missing physiological information is complemented as a zero value.

The generation unit 31 generates display data for visually presenting the condition information or the condition information and the missing information to a user based on the condition information or the condition information and the missing information. The user is, for example, a medical worker or the like. The display data generated based on the condition information and the missing information includes at least one of first character information for presenting the missing information to the user in characters, pattern information for presenting the missing information to the user in a geometric patter, and color information for presenting the missing information to the user in a color. When generating the display data based on the condition information and the missing information, the generation unit 31 generates first display data, second display data, or third display data as the display data. The first display data includes the condition information and the missing information. The second display data includes second character information for presenting the missing information to the user in characters, the condition information, and the missing information. The second character information is generated by the generation unit 31 in response to a predetermined operation on a display screen based on the display data. The third display data includes detailed information on the physiological information corresponding to the missing information. The detailed information on the physiological information corresponding to the missing information is, for example, a measurement time, a measurement result, a score based on the condition determination method, a measurement value related to the missing physiological information, and the like.

The output interface 4 is configured to output an output signal OS corresponding to the condition information. The output interface 4 can transmit the output signal OS to, for example, an external device 20 which is a device different from the condition information generation device 10. The output interface 4 may include a circuit for converting output data into the output signal OS that can be processed by the external device 20, as necessary.

The external device 20 is a display device including a display unit 21, a mobile terminal, or the like. The display unit 21 is, for example, a liquid crystal display, an organic EL display, or the like. When the external device 20 receives the output signal OS from the output interface 4, the external device 20 displays the condition information on the patient P corresponding to the output signal OS on the display unit 21.

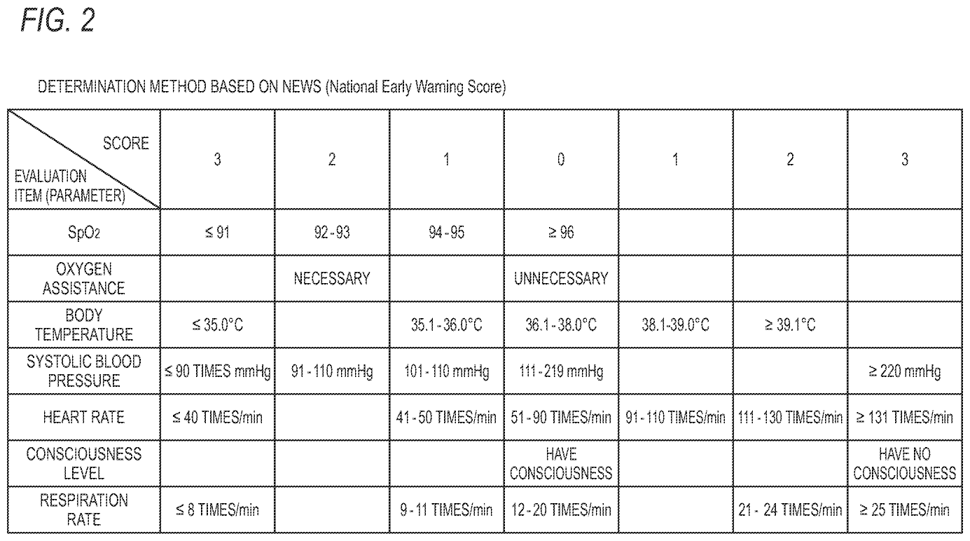

Next, the condition information on the patient P generated by the generation unit 31 will be described in detail with reference to FIGS. 2 to 4. FIG. 2 is a diagram illustrating a reference value of NEWS which is one of the condition determination methods. NEWS is a determination method used for determination of sepsis. When the acquired respiration rate per minute is 8 times or less or 25 times or more, a score related to the respiration rate is 3. When the respiration rate is 21 times or more and 24 times or less, the score related to the respiration rate is 2. When the respiration rate is 9 times or more and 11 times or less, the score related to the respiration rate is 1. When the respiration rate is 12 times or more and 20 times or less, the score related to the respiration rate is 0. In addition, for the remaining parameters, the score is calculated based on a reference value or the like determined by medical guideline or the like. The calculated scores are summed. In the present specification, the total score is referred to as a total score (an example of the condition information). The calculation and addition of the scores are performed by the generation unit 31. In addition, in the present embodiment, the total score is a numerical value, but may be expressed in another expression form such as a percentage.

As the condition determination method related to sepsis, there is another condition determination method such as qSOFA in addition to NEWS. In addition, medical meaning of the reference value and the value of the total score is different for each condition determination method. Therefore, in the present embodiment, the generation unit 31 generates the condition level information (an example of the condition information) based on various scores such as the total score in each determination method and the normalization table.

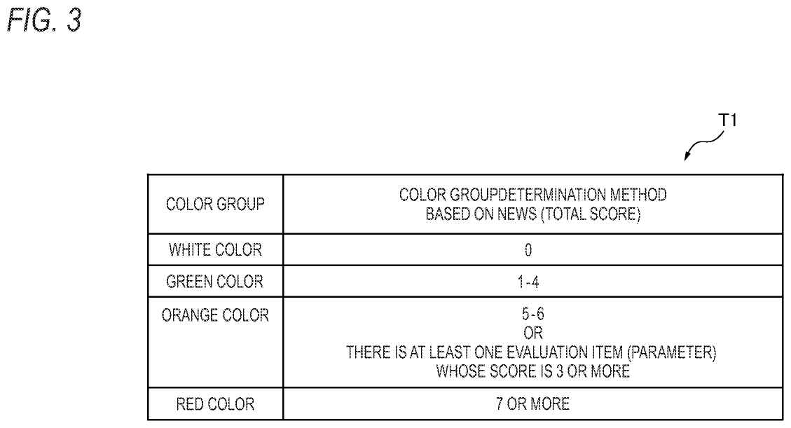

FIG. 3 is a diagram illustrating a normalization table T1 in NEWS. The normalization table T1 is a table for classifying the condition levels of the patient P into four color groups and displaying the four color group. The four color groups in the present embodiment are a white color group, a green color group, an orange color group, and a red color group. The white color group corresponds to a condition level indicating that the patient P is in a normal state. The red color group corresponds to a condition level indicating that the patient P is in an abnormal state and in a dangerous state. The orange color group corresponds to a condition level indicating that the patient P is not in the normal state although not as much as the patient of the red color group. The green color group corresponds to a condition level indicating that the patient P is not in the normal state although not as much as the patients of the orange color group. That is, the condition levels of patients other than the white color group are worse in an order of the green color group, the orange color group, and the red color group, incidentally, the classification of the color groups described here is merely an example. It is needless to say that the classification of the color groups is not limited to this example.

In NEWS, for example, when the total score is 0, the, color group is classified into the white color group. When the total score is 1 or more and 4 or less, the color group is classified into the green color group. When the total score is 5 or more and 6 or less, or when the score of at least one of the evaluation items in NEWS is 3, the color group is classified into the orange color group. When the total score is 7 or more, the color group is classified into the red color group.

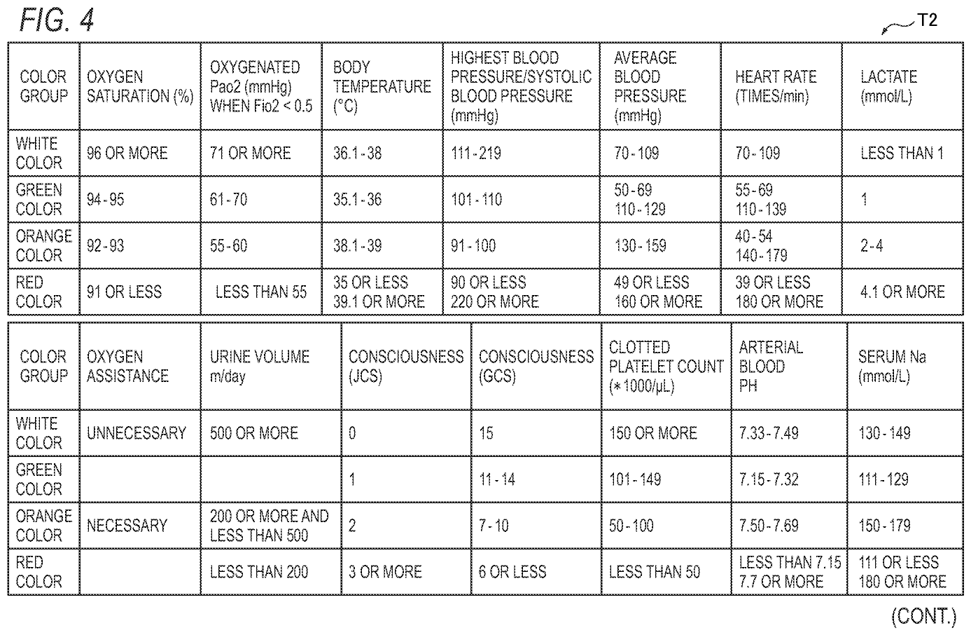

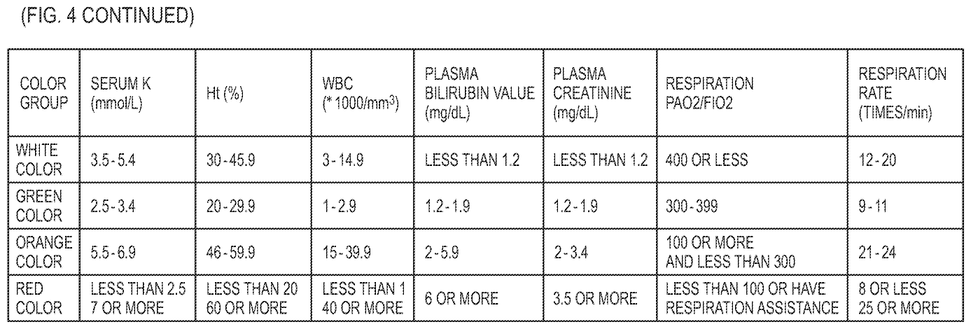

FIG. 4 is a diagram illustrating a normalization table T2 for normalizing each piece of physiological information. As illustrated in FIG. 4, for example, when the respiration rate per minute is 12 times or more and 20 times or less, the color group is classified as the white color group. When the respiration rate is 9 times or more and 11 times or less, the color group is classified into the green color group. When the respiration rate is 21 times or more and 24 times or less, the color group is classified into the orange color group. When the respiration rate is 8 or less and 25 or more, the color group is classified into the red color group.

As illustrated in FIG. 4, for the oxygen assistance, the color group is classified into the orange color group when the oxygen assistance is necessary, and the color group is classified into the white color group when the oxygen assistance is unnecessary, in addition, regarding a urine volume, when the urine volume per day is 500 mL or more, the color group is classified into the white color group. When the urine volume per day is 200 mL or more and less than 500 mL, the color group is classified into the orange color group. When the urine volume per day is less than 200 mL, the color group is classified into the red color group. The other physiological information is classified into four stages, and one color group is associated with each stage.

The above-described classification is performed by the generation unit 31. That is, the generation unit 31 classifies the condition level of the patient P based on the total score or each piece of physiological information, and the normalization table T1 or the normalization table T2 stored in the storage unit 2. For example, in a case where NEWS is used to determine whether a certain patient has sepsis, the generation unit 31 determines into which color group of the four color groups the condition level of the patient P is classified based on the total score and the normalization table T1 stored in the storage unit 2, and generates the condition level information of the patient P corresponding to the determination result.

### First Embodiment

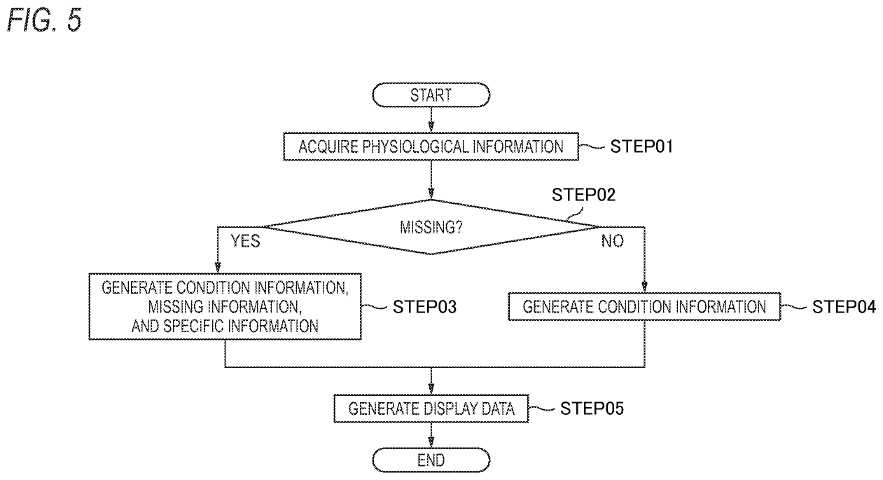

Next, the process performed by the condition information generation device 10 will be described in detail with reference to FIGS. 5 to 10. In the present embodiment, the condition information generation device 10 acquires physiological information of four patients P1 to P4 from 13:00 to 16:30, and determines a condition of each of the patients P1 to P4 based on the acquired physiological information of the patients P1 to P4 and NEWS. The patient P1 is Taro Kohden. The patient P2 is Jiro Kohden. The patient P3 is Saburo Kouden. The patient P4 is Hanako Kouden.

FIG. 5 is a flowchart of a process performed by the condition information generation device 10. The acquisition unit 1 of the condition information generation device 10 acquires the physiological information of the patients P1 to P4 (STEP 01). In the present embodiment, the physiological information of the patient P1 acquired by the acquisition unit 1 from 13:00 to 16:30 is physiological information (SpO₂, oxygen assistance, a body temperature, a systolic blood pressure, a heart rate, a consciousness level, and a respiration rate) that is normally used when executing NEWS, The physiological information of the patient P2 acquired by the acquisition unit 1 from 13:00 to 15:30 is the physiological information normally used when executing NEWS. The physiological information of the patient P2 acquired by the acquisition unit 1 from 15:30 to 16:30 is physiological information other than the body temperature among the physiological information normally used when executing NEWS. The physiological information of the patient P3 acquired by the acquisition unit 1 from 13:00 to 15:30 is the physiological information normally used when executing NEWS. The physiological information of the patient P3 acquired by the acquisition unit 1 from 15:30 to 16:00 is physiological information other than the body temperature among the physiological information normally used when executing NEWS. The physiological information of the patient P3 acquired by the acquisition unit 1 from 16:00 to 16:30 is the physiological information normally used when executing NEWS. The physiological information of the patient P4 acquired by the acquisition unit 1 from 13:00 to 15:30 is the physiological information normally used when executing NEWS. The physiological information of the patient P4 acquired by the acquisition unit 1 from 15:30 to 16:30 is physiological information other than the body temperature and the systolic blood pressure among the physiological information normally used when executing NEWS.

That is, in the present embodiment, there is missing physiological information in the physiological information of the patient P2 acquired by the acquisition unit 1 from 15:30 to 16:30, the physiological information of the patient P3 acquired by the acquisition unit 1 from 15:30 to 16:00, and the physiological information of the patient P4 acquired by the acquisition unit 1 from 15:30 to 16:30. As described above, since the continuous measurement parameter such as the SpO₂ is a parameter for which the data is continuously measured from the sensor attached to the patient P, the data is less likely to be missing. On the other hand, the discontinuous measurement parameters such as the body temperature and the systolic blood pressure are parameters that are measured in a spot manner from the sensor attached to the patient P, and thus are more likely to be missing than the continuous measurement parameters.

The generation unit 31 of the condition information generation device 10 determines, for each of the patients P1 to P4, whether there is missing physiological information in the physiological information of the patients P1 to P4 acquired by the acquisition unit 1 (STEP 02). When there is missing physiological information (YES in STEP 02), the generation unit 31 generates the condition information, the missing information, and the specific information (STEP 03). In the present embodiment, when generating the condition information, the generation unit 31 uses the acquired physiological information for the acquired physiological information and uses a zero value (an example of the complementary physiological information) for the physiological information that is not acquired. In addition, at this time, the missing information generated by the generation unit 31 includes the complementary type information indicating that the missing physiological information is complemented as the zero value. The specific information generated by the generation unit 31 is information for specifying the number of pieces of missing physiological information (one or two pieces in the present embodiment) and the type of the missing physiological information (the body temperature, or the body temperature and the systolic blood pressure in the present embodiment). On the other hand, when there is no missing physiological information (NO in STEP 02), the generation unit 31 generates the condition information (STEP 04).

In the present embodiment, the acquisition unit 1 acquires, for the patient P1, all of the physiological information normally used when executing NEWS. Therefore, the generation unit 31 determines that there is no missing physiological information (NO in STEP 02), and generates the condition information (STEP 04).

On the other hand, for the patients P2 to P4, there is a time zone in which the body temperature, or the body temperature and the systolic blood pressure are not acquired by the acquisition unit 1, among the physiological information normally used when executing the NEWS. Therefore, the generation unit 31 determines that there is missing in the time zone in which the body temperature, or the body temperature and the systolic blood pressure are not acquired (YES in STEP 02), and generates the condition information, the missing information, and the specific information (STEP 03). On the other hand, the generation unit 31 determines that there is no missing in the time zone in which all of the physiological information normally used when executing NEWS is acquired (NO in STEP 02), and generates the condition information (STEP 04).

In the present embodiment, the total score of the patient P1 from 13:00 to 13:30 is 0. The total score of the patient P1 from 13:30 to 14:30 is 1. The total score of the patient P1 from 14:30 to 15:30 is 5. The total score of the patient P1 from 15:30 to 16:30 is 7. The total score of the patient P2 from 13:00 to 13:30 is 0. The total score of the patient P2 from 13:30 to 15:30 is 1. The total score of the patient P2 from 15:30 to 16:30 is 5. The total score of the patient P3 from 13:00 to 14:30 is 0. The total score of the patient P3 from 14:30 to 15:30 is 1. The total score of the patient P3 from 15:30 to 16:00 is 6. The total score of the patient P3 from 16:00 to 16:30 is 7. The total score of the patient P4 from 13:00 to 15:00 is 0. The total score of the patient P4 from 15:00 to 16:30 is 1.

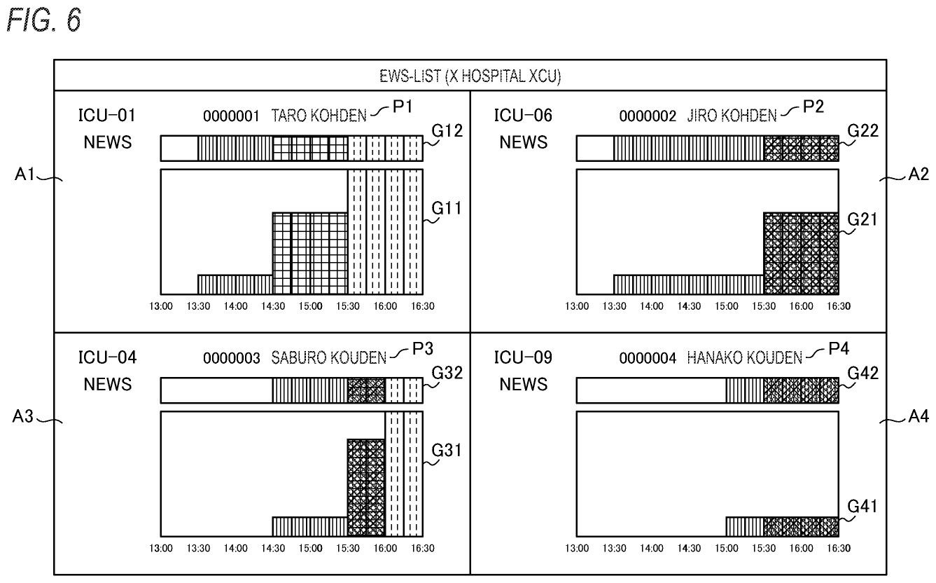

The generation unit 31 generates the display data for visually presenting the condition information, or the condition information and the missing information to the user based on the condition information, or the condition information and the missing information (STEP 05). When the generation unit 31 generates the display data based on the condition information and the missing information, the missing information may include the complementary type information. The generation unit 31 generates any one of the first display data, the second display data, and the third display data as the display data. A first display image based on the first display data is, for example, the display image illustrated in FIG. 6. The first display image is displayed on, for example, the display unit 21 of the external device 20. The first display image is divided into four areas A1 to A4. The area A1 is located at an upper left portion of the first display image. The area A2 is located in an upper right portion of the first display image. The area A3 is located in a lower left portion of the first display image. The area A4 is located in a lower right portion of the first display image. In the present embodiment, the sizes of the areas A1 to A4 are equal to each other, but may be different from each other.

In the area A1, a vertical bar graph G11 and a horizontal bar graph G12 indicating the condition level of the patient P1 (Taro Kohden) from 13:00 to 16:30 are displayed. The vertical bar graph G11 and the horizontal bar graph G12 are colored in accordance with the condition level of the patient P1 determined by NEWS. In the present embodiment, hatching is used instead of color. The hatching corresponding to the white color group is blank. The hatching corresponding to the green color group is vertical lines. The hatching corresponding to the orange color group is horizontal lines. The hatching corresponding to the red color group is broken lines extending in a vertical direction. Since there is no missing physiological information in the physiological information of the patient P1 acquired by the acquisition unit 1 from 13:00 to 16:30, a display mode of the vertical bar graph G11 and the horizontal bar graph G12 is a display mode based on the condition information.

In the area A2, a vertical bar graph G21 and a horizontal bar graph G22 indicating the condition level of the patient P2 (Jiro Kohden) from 13:00 to 16:30 are displayed. Although there is no missing physiological information in the physiological information of the patient P2 acquired by the acquisition unit 1 from 13:00 to 15:30, there is missing physiological information in the physiological information of the patient P2 acquired by the acquisition unit 1 from 15:30 to 16:30. Therefore, the display mode of portions of the vertical bar graph G21 and the horizontal bar graph G22 from 13:00 to 15:30 is a display mode based on the condition information. On the other hand, the display mode of portions from 15:30 to 16:30 is a display mode based on the condition information and the missing information. In the vertical bar graph G21 and the horizontal bar graph G22, portions from 13:00 to 15:30 are only colored corresponding to the condition level of the patient P2. On the other hand, in the vertical bar graph G21 and the horizontal bar graph G22, portions from 15:30 to 16:30 are hatched in a grid-like pattern (an example of a geometric pattern), in addition to being colored corresponding to the condition level of the patient P2. The information corresponding to the grid-like hatching (an example of the pattern information) is information indicating that at least one piece of physiological information among pieces of physiological information normally used when executing NEWS is missing. That is, the information corresponding to the grid-like hatching is based on the missing information. It is needless to say that the display mode based on the missing information is not limited to the grid-like hatching. The display mode based on the missing information may be, for example, hatching of another pattern, or may be colored in another color different from the color corresponding to the condition level of the patient P.

In the area A3, a vertical bar graph G31 and a horizontal bar graph G32 indicating the condition level of the patient P3 (Saburo Kouden) from 13:00 to 16:30 are displayed. Although there is no missing physiological information in the physiological information of the patient P3 acquired by the acquisition unit 1 from 13:00 to 15:30 and from 16:00 to 16:30, but there is missing physiological information in the physiological information of the patient P3 acquired by the acquisition unit 1 from 15:30 to 16:00. Therefore, in the vertical bar graph G31 and the horizontal bar graph G32, the display modes of portions from 13:00 to 15:30 and from 16:00 to 16:30 are display modes based on the condition information. On the other hand, the display mode of portions from 15:30 to 16:00 is a display mode based on the condition information and the missing information. In the vertical bar graph G31 and the horizontal bar graph G32, the portions from 13:00 to 15:30 and from 16:00 to 16:30 are only colored corresponding to the condition level of the patient P3. On the other hand, in the vertical bar graph G31 and the horizontal bar graph G32, the portions from 15:30 to 16:00 are hatched in a grid-like pattern, in addition to being colored corresponding to the condition level of the patient P3.

In the area A4, a vertical bar graph G41 and a horizontal bar graph (342 indicating the condition level of the patient P4 (Hanako Kouden) from 13:00 to 16:30 are displayed. Although there is no missing physiological information in the physiological information of the patient P4 acquired by the acquisition unit 1 from 13:00 to 15:30, there is missing physiological information in the physiological information of the patient P4 acquired by the acquisition unit 1 from 15:30 to 16:30. Therefore, in the vertical bar graph G41 and the horizontal bar graph G42, the display mode of portions from 13:00 to 15:30 is a display mode based on the condition information. On the other hand, the display mode of portions from 15:30 to 16:30 is a display mode based on the condition information and the missing information. In the vertical bar graph G41 and the horizontal bar graph (142, the portions from 13:00 to 15:30 are only colored corresponding to the condition level of the patient P4. On the other hand, in the vertical bar graph G41 and the horizontal bar graph G42, the portions from 15:30 to 16:30 are hatched in a grid-like pattern, in addition to being colored corresponding to the condition level of the patient P4.

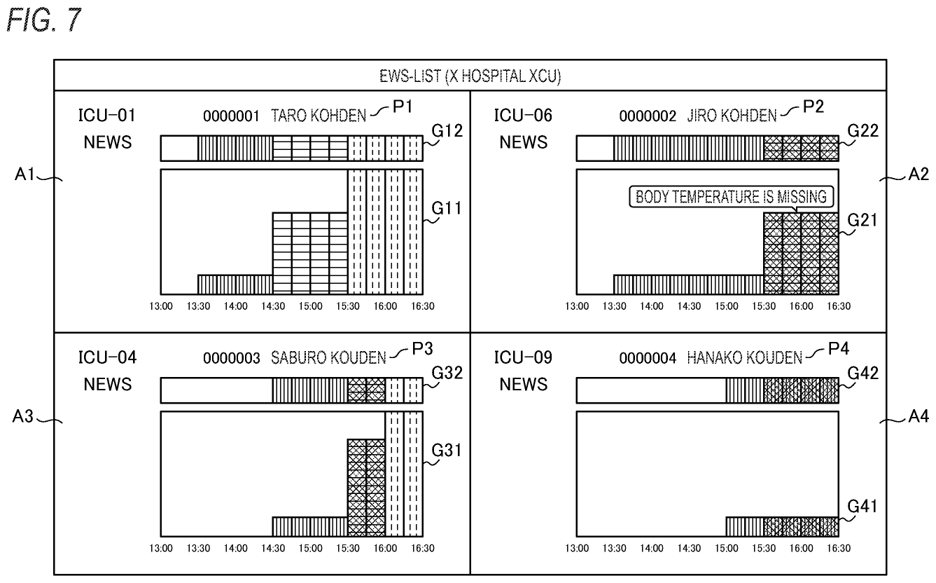

For example, when the user moves a mouse cursor to a portion of the vertical bar graph G21 that is hatched in a grid-like pattern, the generation unit 31 generates the second display data including the second character information, the condition information, and the missing information as the display data based on the condition information and the missing information. A second display image based on the second display data is a display image illustrated in FIG. 7. The second display image is displayed on, for example, the display unit 21 of the external device 20. As illustrated in FIG. 7, in the second display image, a message an example of the second character information) including specific information on the patient P2, “body temperature is missing”, is displayed in a form of a tooltip in the vicinity of the grid-like hatched portion of the vertical bar graph G21. However, the message does not have to include the specific information on the patient P2. Note that the message may be displayed in the vicinity of a portion of the horizontal bar graph G22 that is hatched in a grid-like pattern, when the user moves the mouse cursor to the grid-like hatched portion of the horizontal bar graph G22. In addition, the tooltip is a tooltip that displays, in a text format, an explanation and the like about a pointed object to the user when the user points a cursor or a pointer on the object (in the present embodiment, the vertical bar graph G21 or the horizontal bar graph G22), that is, when the cursor or the pointer is superimposed on the object.

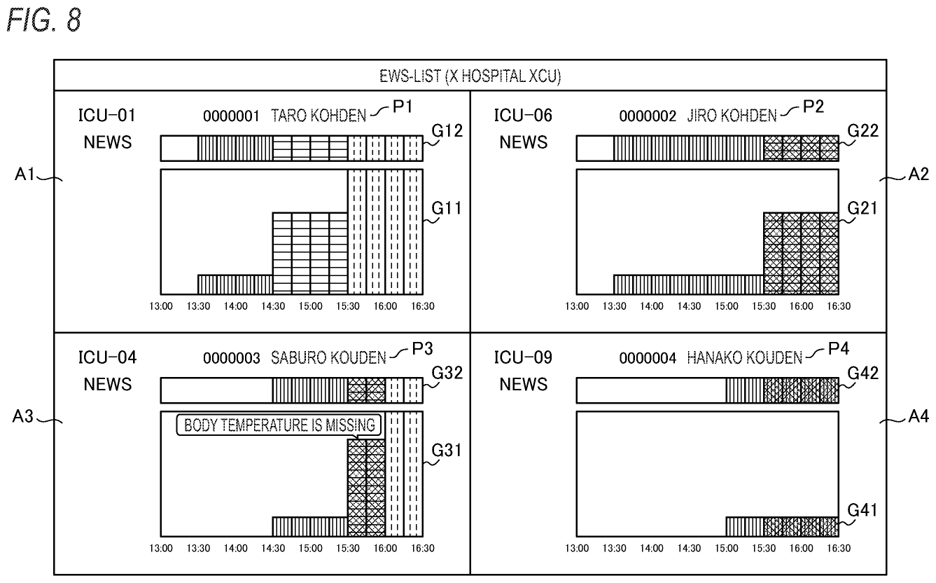

When the user moves the mouse cursor to a portion of the vertical bar graph G31 that is hatched in a grid-like pattern, the generation unit 31 generates the second display data, including the second character information, the condition information, and the missing information as the display data based on the condition information and the missing information. The second display image based on the second display data is a display image illustrated in FIG. 8. As illustrated in FIG. 8, in the second display image, a message (an example of the second character information) including specific information on the patient P3, “body temperature is missing”, is displayed in the form of a tooltip in the vicinity of a portion of the vertical bar graph G31 that is hatched in a grid-like pattern. However, the message does not have to include the specific information on the patient P3. Note that the message may be displayed in the vicinity of a portion of the horizontal bar graph G32 that is hatched in a grid-like pattern, when the user moves the mouse cursor to the grid-like hatched portion of the horizontal bar graph G32.

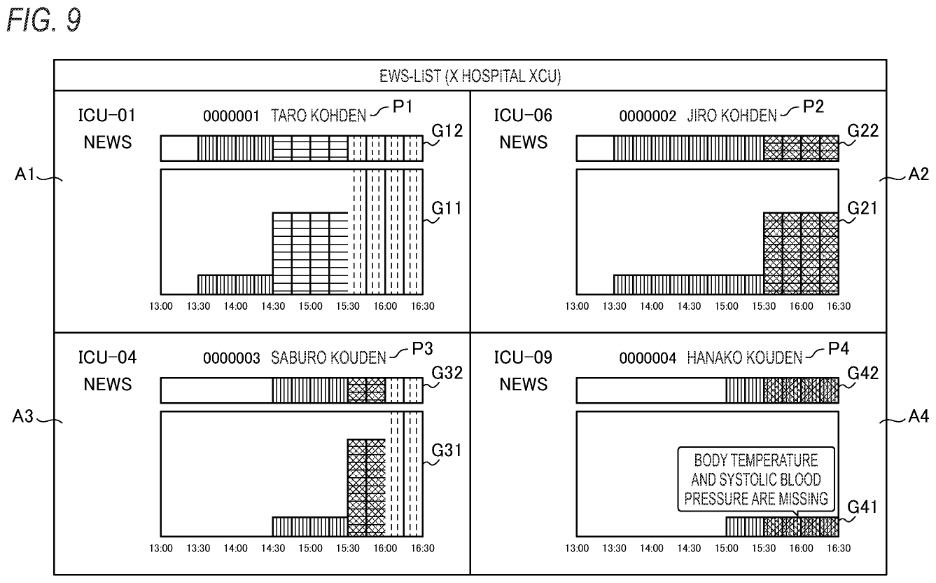

When the user moves the mouse cursor to a portion of the vertical bar graph G41 that is hatched in a grid-like pattern, the generation unit 31 generates the second display data including the second character information, the condition information, and the missing information as the display data based on the condition information and the missing information. The second display image based on the second display data is a display image illustrated in FIG. 9. As illustrated in FIG. 9, in the second display image, a message (an example of the second character information) including specific information on the patient P4, “body temperature and systolic blood pressure are missing”, is displayed in the form of a tooltip in the vicinity of a portion of the vertical bar graph G41 that is hatched in a grid-like pattern. However, the message does not have to include the specific information on the patient P4. Note that the message may be displayed in the vicinity of a portion of the horizontal bar graph G42 that is hatched in a grid-like pattern, when the user moves the mouse cursor to the grid-like hatched portion of the horizontal bar graph G42.

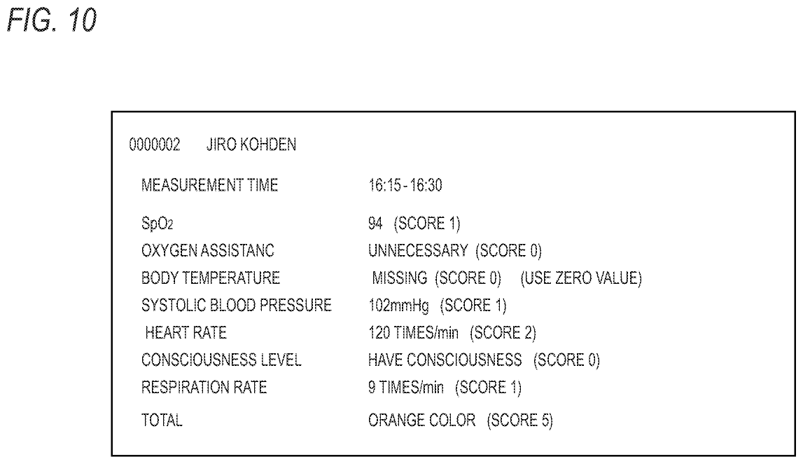

For example, when the user clicks a portion of the vertical bar graph G21 corresponding to 16:15 to 16:30, the generation unit 31 generates the third display data including the detailed information as the display data. A third display image based on the third display data generated at this time is a display image illustrated in FIG. 10. The third display image is displayed on, for example, the display unit 21 of the external device 20. As illustrated in FIG. 10, in the third display image, a patient identification number, a patient name, a measurement time, the physiological information normally used when executing NEWS, a measurement result (measurement value or the like) of the physiological information, a score, a total score, and a color group corresponding to a condition level are displayed. Therefore, the user can confirm in detail the physiological information and the like of the patient P2 from 16:15 to 16:30 by viewing the third display image illustrated in FIG. 10. In addition, in the third display image illustrated in FIG. 10, a message indicating that the missing body temperature has been complemented with a zero value is displayed. Therefore, the user recognizes that the missing body temperature is complemented with the zero value by viewing the third display image illustrated in FIG. 10.

According to the condition information generation device 10 configured as described above, the generation unit 31 generates the condition information based on the acquired physiological information and a determination method such as NEWS. In addition, the generation unit 31 determines whether there is missing physiological information in the acquired physiological information with respect to the physiological information normally used when a determination method such as NEWS is executed, and when there is missing physiological information, the generation unit 31 generates the missing information indicating that there is missing. That is, the generation unit 31 generates the condition information and the missing information when there is missing physiological information in the acquired physiological information with respect to the physiological information normally used when the determination method such as NEWS is executed. Therefore, the user can easily grasp the condition information and the accuracy of the condition information by using the condition information and the missing information.

According to the condition information generation device 10 configured as described above, the generation unit 31 generates the display data for visually presenting the condition information and the missing information to the user based on the condition information and the missing information. Therefore, the user can visually recognize the condition information and the missing information by such display data. As a result, the user can intuitively recognize the condition information and the missing information.

According to the condition information generation device 10 configured as described above, the display data includes the pattern information for presenting the missing information to the user in a geometric pattern (in the present embodiment, the grid-like hatching). Therefore, since the missing information is presented to the user in a geometric pattern, the user can intuitively recognize the missing information.

According to the condition information generation device 10 configured as described above, the generation unit 31 generates, as the display data, the first display data including the condition information and the missing information or the second display data including the second character information, the condition information, and the missing information. Therefore, for example, when the user wants to confirm only the condition information and the missing information, the user can confirm the first display data, and when the user wants to confirm the second character information together, the user can confirm the desired information without excess or deficiency just by confirming the second display data.

According to the condition information generation device 10 configured as described above, the display data includes the condition information and the missing information on the plurality of patients P1 to P4. Therefore, by using such display data, the user can grasp the condition information and the missing information on the plurality of patients P1 to P4 in a list. In the present embodiment, the body temperatures of the patients P2 to P4 are missing from 15:30 to 16:00. For example, in a case where the patients P2 to P4 are in the same hospital room, the user can estimate that there is a possibility that the measurement of the body temperature by the round diagnosis is not appropriately performed in the hospital room in which the patients P2 to P4 are present during a period from 15:30 to 16:00.

According to the condition information generation device 10 configured as described above, the generation unit 31 generates, as the display data, the first display data including the condition information and the missing information or the third display data including the detailed information on the physiological information corresponding to the missing information. Therefore, for example, by confirming the third display data after confirming the first display data, the user can confirm the detailed information related to the physiological information corresponding to the missing information after recognizing that there is missing physiological information in the specific condition information.

According to the condition information generation device 10 configured as described above, the generation unit 31 generates the specific information for specifying the physiological information corresponding to the missing information. Therefore, the user can easily specify the physiological information that has not been acquired by using the physiological information as the specific information.

According to the condition information generation device 10 configured as described above, since the specific information is information for specifying the number and the type of physiological information corresponding to the missing information, the user can grasp the number and the type of the missing physiological information by using the specific information. As a result, the user can estimate whether the accuracy of the condition information is high or low.

In addition, according to the condition information generation device 10 configured as described above, when there is missing physiological information in the acquired physiological information with respect to the physiological information normally used when the determination method such as the NEWS is executed, the generation unit 31 generates the condition information based on the acquired physiological information, the zero value as the complementary physiological information, the determination method of the NEWS, or the like. Further, at this time, the generation unit 31 generates the missing information including the complementary type information on the type of complement. Therefore, the user can easily recognize how the physiological information that has not been acquired has been complemented.

### Second Embodiment

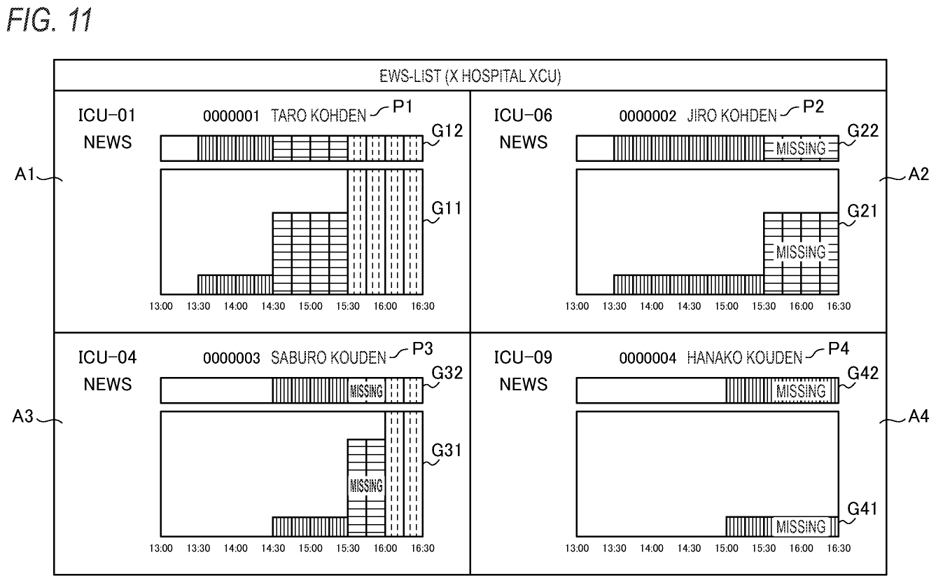

Next, a second embodiment will be described with reference to FIG. 11. FIG. 11 is a diagram illustrating a first display screen based on the first display data in the present embodiment. In the present embodiment, the same components as those of the first embodiment are denoted by the same reference numerals, and the description of the overlapping portions will be appropriately omitted. Also in the second embodiment, the condition information generation device 10 performs the process illustrated in FIG. 5. Also in the second embodiment, the condition information generation device 10 acquires the physiological information of the patients P 1 to P4 from 13:00 to 16:30, and determines the condition of each of the patients P1 to P4 based on the acquired physiological information of the patients P1 to P4 and NEWS. The second embodiment is different from the first embodiment in that, as a display mode for indicating that at least one piece of physiological information among the pieces of physiological information normally used when executing NEWS is missing, a message “missing” (an example of the first character information) is displayed instead of grid-like hatching. It is needless to say that a message for indicating that at least one piece of physiological information among the pieces of physiological information normally used when executing NEWS is missing is not limited to “missing”.

In the present embodiment, an acquisition state of the physiological information of the patients P1 to P4 is the same as that of the first embodiment. That is, since there is no missing physiological information in the physiological information of the patient P1 acquired by the acquisition unit 1 from 13:00 to 16:30, as illustrated in FIG. 11, the message “missing” is not displayed in the area A1. On the other hand, a part of the physiological information of the patients P2 to P4 acquired by the acquisition unit 1 from 13:00 to 16:30 is missing. For this reason, in the area A2, a message “missing” is displayed in the portions of the vertical bar graph G21 and the horizontal bar graph G22 from 15:30 to 16:30. In the area A3, a message “missing” is displayed in the portions of the vertical bar graph G31 and the horizontal bar graph G32 from 15:30 to 16:00. In the area A4, a message “missing” is displayed in the portions of the vertical bar graph G41 and the horizontal bar graph G42 from 15:30 to 16:30. The message “missing” may be displayed on only one of the vertical bar graph G21 and the horizontal bar graph G22, only one of the vertical bar graph G31 and the horizontal bar graph G32, and only one of the vertical bar graph G41 and the horizontal bar graph G42.

Also in the present embodiment, the condition information generation device 10 can achieve the same effects as those of the first embodiment.

According to the condition information generation device 10 configured as described above, the display data includes the first character information for presenting the missing information to the user in characters. Specifically, in the present embodiment, a message “missing” (an example of the first character information) is displayed in a portion where there is missing physiological information. Therefore, since the missing information is presented to the user in characters, the user can intuitively recognize the missing information.

### Third Embodiment

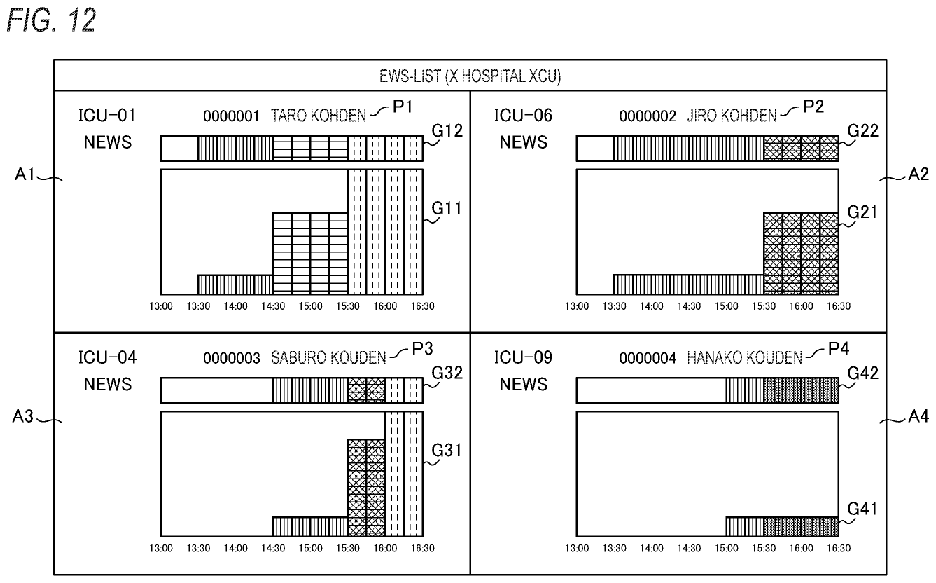

Next, a third embodiment will be described with reference to FIG. 12. FIG. 12 is a diagram illustrating the first display screen based on the first display data in the present embodiment, In the present embodiment, the same components as those of the first embodiment are denoted by the same reference numerals, and the description of the overlapping portions will be appropriately omitted. Also in the third embodiment, the condition information generation device 10 performs the process illustrated in FIG. 5. In the third embodiment, the condition information generation device 10 acquires the physiological information of the patients P1 to P4 from 13:00 to 16:30, and determines the condition of each of the patients P1 to P4 based on the acquired physiological information of the patients P1 to P4 and NEWS. The third embodiment is different from the first embodiment in that a display mode for indicating that at least one piece of physiological information among the pieces of physiological information normally used when executing NEWS is missing is changed according to the number of pieces of missing physiological information.

In the present embodiment, there is no missing physiological information in the physiological information of the patient P1 acquired by the acquisition unit 1 from 13:00 to 16:30. However, there is missing physiological information in the physiological information of the patient P2 acquired by the acquisition unit 1 from 15:30 to 16:30, the physiological information of the patient P3 acquired by the acquisition unit 1 from 15:30 to 16:00, and the physiological information of the patient P4 acquired by the acquisition unit 1 from 15:30 to 16:30. In the present embodiment, the physiological information of the patient P2 acquired by the acquisition unit 1 of the condition information generation device 10 from 15:30 to 16:30 is physiological information other than the body temperature among the physiological information (SpO₂, oxygen assistance, a body temperature, a systolic blood pressure, a heart rate, a consciousness level, and a respiration rate) that is normally used when executing NEWS. The physiological information of the patient P3 acquired by the acquisition unit 1 of the condition information generation device 10 from 15:30 to 16:00 is physiological information other than the body temperature among the physiological information normally used when executing NEWS. The physiological information of the patient P4 acquired by the acquisition unit 1 of the condition information generation device 10 from 15:30 to 16:30 is physiological information other than the body temperature and the systolic blood pressure among the physiological information normally used when executing NEWS.

The generation unit 31 determines that the body temperature is missing in the physiological information of the patient P2 acquired from 15:30 to 16:30, and generates the specific information based on the determination. At this time, the specific information generated by the generation unit 31 is information for specifying that the number of pieces of missing physiological information is one and the type of missing physiological information is the body temperature. The generation unit 31 determines that the body temperature is missing in the physiological information of the patient P3 acquired from 15:30 to 16:00, and generates the specific information based on the determination. At this time, the specific information generated by the generation unit 31 is information for specifying that the number of pieces of missing physiological information is one and the type of missing physiological information is the body temperature, The generation unit 31 determines that the body temperature and the systolic blood pressure are missing in the physiological information of the patient P4 acquired from 15:30 to 16:30, and generates the specific information based on the determination. The specific information generated by the generation unit 31 at this time is information for specifying that the number of pieces of missing physiological information is two and the type of missing physiological information is the body temperature and the systolic blood pressure.

In the present embodiment, the generation unit 31 generates the display data based. on the condition information, or the condition information, the missing information, and the specific information. As illustrated in FIG. 12, a portion where one piece of physiological information is missing (for example, the portions of the vertical bar graph G21 and the horizontal bar graph G22 from 15:30 to 16:30) is hatched in a grid-like pattern in addition to being colored corresponding to the condition level of the patient P. On the other hand, a portion where the two pieces of physiological information are missing (for example, the portions of the vertical bar graph G41 and the horizontal bar graph G42 from 15:30 to 16:30) is hatched with dots in addition to being colored corresponding to the condition level of the patient P. It is needless to say that the display mode in the portion where one piece of physiological information is missing and the display mode in the portion where two pieces of physiological information are missing are not limited to the grid-like hatching and the dot hatching. The display mode in the portion in which one piece of physiological information is missing and the display mode in the portion in which two pieces of physiological information are missing may be, for example, hatching of another pattern, or may be colored in a color different from the color corresponding to the condition level of the patient P.

As described above, in the present embodiment, the display mode for indicating that at least one piece of physiological information among the pieces of physiological information normally used when executing NEWS is missing is changed according to the number of pieces of missing physiological information. Therefore, the user can easily recognize the presence or absence of missing of the physiological information and the number of pieces of missing physiological information simply by viewing a second display screen based on the second display data.

Also in the present embodiment, the condition information generation device 10 can achieve the same effects as those of the first embodiment.

### Fourth Embodiment

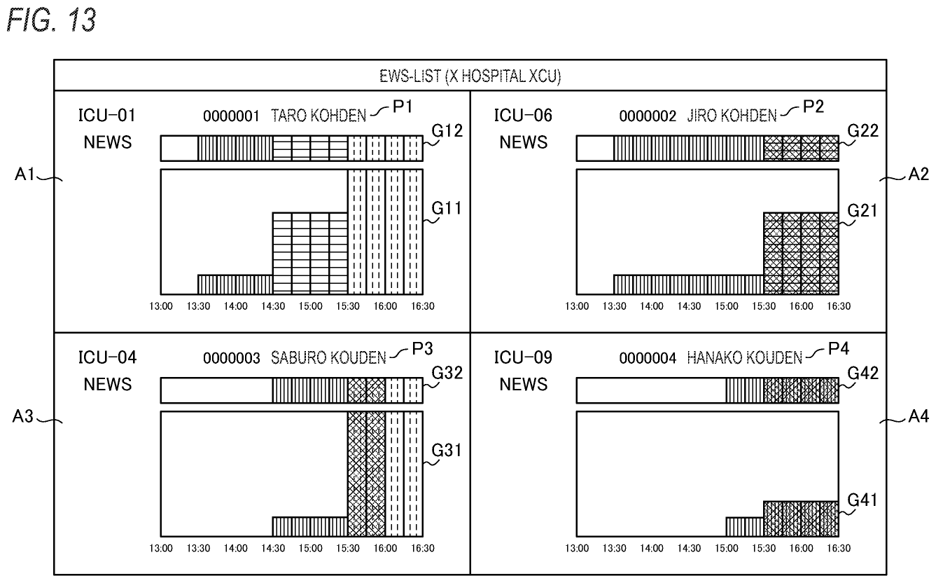

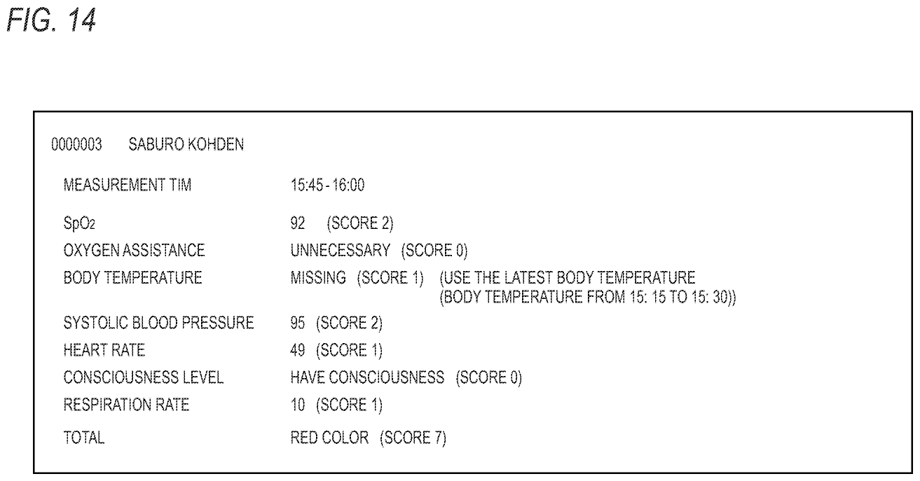

Next, a fourth embodiment will be described with reference to FIGS. 13 and 14. FIG. 13 is a diagram illustrating the first display screen based on the first display data in the present embodiment. FIG. 14 is a diagram illustrating a third display screen based on the third display data in the present embodiment. In the present embodiment, the same components as those of the first embodiment are denoted by the same reference numerals, and the description of the overlapping portions will be appropriately omitted. Also in the fourth embodiment, the condition information generation device 10 performs the process illustrated in FIG. 5.

In the present embodiment, the generation unit 31 generates the condition information for the missing physiological information using the latest physiological information among the physiological information acquired so far. The present embodiment is different from the first embodiment in this respect. In the present embodiment, it is assumed that the body temperature of the patient P2 from 15:15 to 15:30 is 36.5° C. (score 0). That is, the generation unit 31 generates the condition information assuming that the body temperature of the patient P2 from 15:30 to 16:30 is 36.5° C. (score 0), It is assumed that the body temperature of the patient P3 from 15:15 to 15:30 is 35.5° C. (score 1). That is, the generation unit 31 generates the condition information assuming that the body temperature of the patient P3 from 15:30 to 16:00 is 35.5° C. (score 1). It is assumed that the body temperature of the patient P4 from 15:15 to 15:30 is 36.5° C. (score 0) and the systolic blood pressure is 105 mmHg (score 1). That is, the generation unit 31 generates the condition information assuming that the body temperature of the patient P4 is 36.5° C. (score 0) and the systolic blood pressure is 105 mmHg (score 1) from 15:30 to 16:30.

In the present embodiment, the total score of the patient P2 from 15:30 to 16:30 is 5. The total score of the patient P3 from 15:30 to 16:00 is 7. The total score of the patient P4 from 15:30 to 16:30 is 2. Therefore, the first display image based on the first display data generated by the generation unit 31 in the present embodiment is a display image illustrated in FIG. 13.

For example, when the user clicks a portion of the vertical bar graph G31 corresponding to 15:45 to 16:00, the generation unit 31 generates the third display data including detailed information as the display data. The third display image based on the third display data generated at this time is a display image illustrated in FIG. 14. As illustrated in FIG. 14, in the third display image, in addition to a patient identification number, a patient name, a measurement time, the physiological information normally used when executing NEWS, a measurement result (measurement value or the like) of the physiological information, a score, the total score, and the color group corresponding to the condition level, the type of the complemented physiological information and the measurement value of the complemented physiological information are displayed. That is, in the third display image illustrated in FIG. 14, a message indicating that the missing body temperature is complemented with the latest body temperature of the patient P3, that is, the body temperature (35.5° C.) of the patient P3 from 15:15 to 15:30 is displayed.

As described above, in the present embodiment, the user can confirm in detail the physiological information and the like of the patient P3 from 15:45 to 16:00 by viewing the third display image, and can also confirm the type of the complemented physiological information and the details of the measurement value of the complemented physiological information. Specifically, for example, the user can easily recognize that the missing physiological information among the physiological information of the patient P3 from 15:45 to 16:00 is the body temperature and that the body temperature of the patient P3 from 15:45 to 16:00 is complemented by the body temperature of the patient P3 from 15:15 to 15:30 by viewing the third display image illustrated in FIG. 14.

Also in the present embodiment, the condition information generation device 10 can achieve the same effects as those of the first embodiment.

The above embodiments are intended to facilitate understanding of the presently disclosed subject matter, and are not intended to limit the presently disclosed subject matter. The presently disclosed subject matter can be modified and improved without departing from the gist thereof.

In the embodiments described above, in the vertical bar graphs G21, G31, and G41 and the horizontal bar graphs G22, G32, and G42, portions where physiological information is missing are hatched in a grid-like pattern or hatched with dots, but the presently disclosed. subject matter is not limited thereto. The grid-like hatching or the dot hatching may be applied to only one of the vertical bar graph G21 and the horizontal bar graph G22, only one of the vertical bar graph G31 and the horizontal bar graph G32, and only one of the vertical bar graph G41 and the horizontal bar graph G42.

In the third embodiment, an example has been described in which the display mode for indicating that at least one piece of physiological information among pieces of physiological information normally used when executing NEWS is missing is changed according to the number of pieces of missing physiological information, but the presently disclosed subject matter is not limited thereto. The display mode for indicating that at least one piece of physiological information among pieces of physiological information normally used when executing NEWS is missing may be changed according to, for example, the type of missing physiological information or the number and types of missing physiological information. In this case, the user can easily recognize the presence or absence of missing of the physiological information and the type of the missing physiological information, or the presence or absence of missing of the physiological information and the number and types of the missing physiological information, simply by viewing the second display screen based on the second display data.

In the embodiments described above, as the display mode for visually presenting the missing information to the user, the grid-like hatching, the dot hatching, or the message “missing” is adopted, but the present disclosure is not limited thereto. The display mode for visually presenting the missing information to the user may be, for example, a display mode in which the portion in which the physiological information is missing is colored differently from the portion in which the physiological information is not missing, among the vertical bar graphs G21, G31, and G41 and the horizontal bar graphs G22, G32, and G42. In this case, the display data generated by the condition information generation device 10 includes the color information for presenting the missing information to the user in a color. In addition, the display data may include two pieces of information among the character information, the first pattern information, and the color information, or all of these pieces of information (that is, the first character information, the pattern information, and the color information).

In the embodiments described above, the first display image, the second display image, and the third display image are displayed on the display unit 21 of the external device 20, but may be displayed on a display unit included in the condition information generation device 10, for example. That is, the presently disclosed subject matter is also applicable to a condition information generation device in which the condition information generation device 10 and the display unit 21 are integrated.

In the embodiments described above, the specific information is information for specifying the number of pieces of missing physiological information and the type of missing physiological information, but may be information for specifying only one of the number of pieces of missing physiological information and the type of missing physiological information.

In the embodiments described above, the conditions of the patients P1 to P4 are determined using NEWS, but the presently disclosed subject matter can also be applied to a case where another condition determination method such as qSOFA is used.

In the embodiments described above, the display data (the first display data, the second display data, and the third display data) includes the condition information and the missing information on a plurality of patients (the patients P1 to P4), but may include only the condition information and the missing information on one patient. That is, the presently disclosed subject matter is also applicable to a display screen on which condition information and the like on one patient are displayed.

In the embodiments described above, the generation unit 31 can generate the first display data, the second display data, and the third display data, but the presently disclosed subject matter is not limited thereto. For example, the generation unit 31 may be capable of generating only the first display data, may be capable of generating only the first display data and the second display data, or may be capable of generating only the first display data and the third display data. The condition information generation device 10 may he mounted on a measurement device such as a so-called bedside monitor or may he realized as a function of a central monitor that acquires data from the bedside monitor. In addition, the condition information generation device 10 may be implemented as a function of a server device that acquires information on a patient from an electronic medical record, a bedside monitor, or the like.

The display mode in the third embodiment described above may include a label display for intuitively recognizing the number of pieces of missing physiological information. The label display is, for example, a legend display indicating a display mode corresponding to the number of pieces of missing physiological information. When such label display is included, the user can intuitively and quickly recognize the number of pieces of missing physiological information based on the label display.

What is claimed is:

1. A condition information generation device comprising:
an acquisition unit configured to acquire physiological information of a patient;
a storage unit in which a determination method for determining a condition of the patient is stored; and
a generation unit configured to generate condition information on the condition of the patient based on the acquired physiological information and the determination method,
wherein the generation unit determines whether there is missing physiological information in the acquired physiological information with respect to physiological information normally used when the determination method is executed, and generates missing information indicating that there is missing physiological information in a case where there is missing.

2. The condition information generation device according to claim 1,
wherein the generation unit generates display data for visually presenting the condition information and the missing information to a user based on the condition information and the missing information.

3. The condition information generation device according to claim 2,
wherein the display data includes at least one of first character information for presenting the missing information to the user in characters, pattern information for presenting the missing information to the user in a geometric pattern, and color information for presenting the missing information to the user in a color.

4. The condition information generation device according to claim 2,
wherein the generation unit generates, as the display data,
first display data including the condition information and the missing information, or
second display data including second character information generated in response to a predetermined operation on a display screen based on the display data, which is information for presenting the missing information to the user in characters, the condition information, and the missing information.

5. The condition information generation device according to claim 2,
wherein the display data includes the condition information and the missing information on a plurality of the patients.

6. The condition information generation device according to claim 2,
wherein the generation unit generates, as the display data, the first display data including the condition information and the missing information, or third display data including detailed information related to physiological information corresponding to the missing information.

7. The condition information generation device according to claim 1,
wherein the generation unit generates specific information for specifying the physiological information corresponding to the missing information.

8. The condition information generation device according to claim 7,
wherein the specific information is information for specifying at least one of a number and a type of physiological information corresponding to the missing information.

9. The condition information generation device according to claim 1,
wherein the generation unit generates the condition information based on the acquired physiological information, complementary physiological information for complementing the missing physiological information, and the determination method, in the case where there is missing physiological information, and
wherein the generation unit generates the missing information including complementary type information on a type of complement.
